(19) United States
(12) Patent Application Publication
(10) Pub. No.: US 2026/0078087 A1
(43) Pub. Date: Mar. 19, 2026

(54) ORGANIC DISELENIDE COMPOSITION

(71) Applicant: SelenBio, Inc., Austin, TX (US)

(72) Inventor: Grant Proulx, Maitland, FL (US)

(21) Appl. No.: 18/870,903

(22) PCT Filed: Nov. 22, 2022
(86) PCT No.: PCT/US2022/050745

§ 371 Date: Dec. 2, 2024

Related U.S. Application Data
(63) Continuation-in-part of application No. PCT/US2022/031687, filed on Jun. 1, 2022.

(51) Int. Cl. C07C 391/00; A01N 55/00; A01P 1/00

(52) U.S. Cl. CPC C07C 391/00; A01N 55/00; A01P 1/00

(57) ABSTRACT

A novel organic diselenide composition comprises 1,2-bis(4-vinylbenzyl) diselane. The organic diselenide composition may be incorporated with one or more polymers through a chemical bonding process for utilization as an agent against infectious, disease-causing agents.

### I. RELATED APPLICATIONS

This application is a continuation-in-part of PCT/US2022/031687 filed on Jun. 1, 2022, which claims the benefit of U.S. Provisional Application No. 63/195,322, filed on Jun. 1, 2021.

### II. FIELD OF THE INVENTION

The present application discloses and describes a novel organic diselenide composition.

### III. BACKGROUND OF THE INVENTION

In the aftermath of the COVID-19 viral outbreak in late 2019, and which persists presently at various locations across the planet, many strategies have been employed and deployed in an attempt to stem the reach and effects of the pandemic. The need for more effective means to prophylactically address such infection disease transmission remains evident. Because infectious disease(s), including sources such as bacteria, viruses, pathogenic fungi, protozoa, and the like, pose significant threats against many living organisms, and especially humans, the concern and attention toward public health issues has been amplified.

In particular, hospitals and health-care facilities wage a persistent battle against infectious agents. Antibiotic resistant bacteria such as well-publicized incidents with MRSA (Methicillin-resistant Staphylococcus aureus), flesh-eating bacteria (usually Group A Streptococci (Streptococcus pyogenes)), as well as antibiotic resistant strains of bacteria that cause pneumonia, diarrhea, meningitis, tuberculosis, and strep-throat remain a persistent threat to those that seek medical treatment in such facilities. And, these threats persistent against the most health-vulnerable, including the very young, the elderly, and those with compromised immune systems.

As a result of widespread public concern connected with infectious disease, antimicrobial treatment of materials such as agents, fabrics, fibers, and polymers have emerged as possible alternatives in preventing the colonization and/or transfer of such infectious disease. While the demand for such antimicrobial articles remains high, relatively few types of such articles are available, and not all of those available are both effective against a broad spectrum of pathogens and capable of sustained antimicrobial activity without being released into the environment or eventually inactivated over time through physical and/or chemical degradation.

Accordingly, there is a need for an improved selenium-based biocidal composition(s), system(s), and/or method(s). More particularly, there is a need for biocidal agents that both avoid the formation of resistant microbes and that can be adapted for use in manufacturing materials, which overcome the disadvantages and defects of the prior art, especially regarding the incorporation of selenium-based biocidal formulations with materials and substrates of various forms or types.

Selenium-based compositions have been known for some time to provide the biocidal properties desired for more effective control of infectious disease. For example, selenium disulfide was described as having great potential therapeutic and fungicidal value in which the product obtained by processes such as those described in U.S. Pat. Nos. 1,860,154 and 1,860,336 issued to B. W. Nordlander (issued in 1932). Further improvements of selenium disulfide were disclosed in U.S. Pat. No. 2,644,669 (issued to Baldwin in 1954) describing fungicidal and bactericidal properties as part of an antidandruff shampoo. U.S. Pat. No. 4,496,559 (Henderson et al.), discloses certain 2-seleno-pyridine-N-oxide compounds and derivatives useful as agricultural fungicides and bactericides, including diselenide-based compounds. Likewise, in a published application (WO2000049868A1 by Arch Chemicals), novel selenium analogs to 2-pyridone and 2-pyrithione compounds are described as fungicidal compounds suitable in shampoos and skin care medicaments to combat dry skin.

In US20080031931, an antimicrobial article includes a substrate and an inorganic or organic selenium compound. The inorganic or organic selenium compound is present in an amount sufficient to treat or prevent the growth or spread of an infectious agent through cutaneous contact of the antimicrobial article with a subject. In particular embodiments, the organoselenium compound has the structure: R—Se, where Se is selenium and R is any organic chemical group. In other embodiments, the organoselenium compound has the structure: R—Se—Se—R′, where Se are selenium atoms, and R and R′ are each, independently, any organic chemical group.

Accordingly, and despite the evolution of organic selenium discovery, and specifically with regard to organic diselenides, there is a need for new synthesis and processes for generating organic diselenide compounds and/or need for new compositions suitable for commercial use.

### IV. SUMMARY OF THE INVENTION

A novel organic diselenide composition comprises 1,2-bis(4-vinylbenzyl) diselane as represented by the chemical structure below:

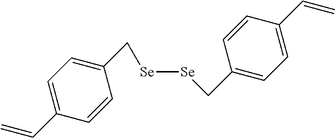 [SMILES: C=Cc1ccc(C[Se][Se]Cc2ccc(C=C)cc2)cc1]

A method for preventing and/or treating a surface with a biocidal agent comprising an organic diselenide having the following formula:

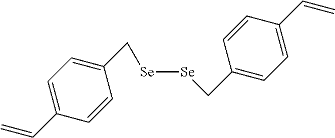 [SMILES: C=Cc1ccc(C[Se][Se]Cc2ccc(C=C)cc2)cc1]

A composition for incorporation with one or more polymers through a chemical bonding process, the composition including one or more polymers as a substrate supporting an organic diselenide having the following formula:

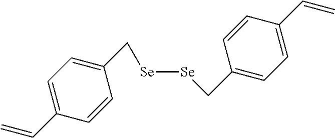 [SMILES: C=Cc1ccc(C[Se][Se]Cc2ccc(C=C)cc2)cc1]

### V. DETAILED DESCRIPTION OF THE EMBODIMENT(S)

It will be readily understood that the components of the present invention, as generally described and illustrated in the figures herein, may be arranged and designed in a wide variety of different configurations. Thus, the following detailed description of the embodiments, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

The presently disclosed and claimed inventive concept(s) is an organic diselenide composition for 1,2-bis(4-vinylbenzyl) diselane as represented by the chemical structure below:

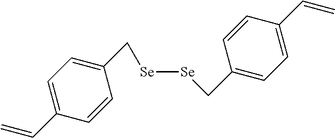 [SMILES: C=Cc1ccc(C[Se][Se]Cc2ccc(C=C)cc2)cc1]

1,2-bis(4-vinylbenzyl) diselane may be prepared by utilizing a closed vessel and introducing a volume of water to solid selenium, thereby forming a selenium slurry in water. Once the selenium slurry is produced, a base is introduced into the vessel. In addition, a surfactant is introduced. Thereafter, a reducing agent is introduced into the vessel, further promoting reducing reaction conditions and encouraging the reduction of solid selenium. Along with the cation(s) from the base, an intermediate compound is produced comprising two elements: a metal ion and diselenide. A reducing agent is used to promote the formation of diselenide ion.

Thereafter, a volume of an organic halide (R-X) is introduced into the vessel to react with the metal ion and diselenide intermediate, thereby encouraging and promoting the formation of a stable organo-diselenide (1,2-bis(4-vinylbenzyl) diselane) material that can be separated from the reaction solution. The yield may be isolated by filtration.

1,2-bis(4-vinylbenzyl) diselane is envisioned as suitable for use through incorporation with one or more polymers through a chemical bonding process. Through the chemical bonding process, 1,2-bis(4-vinylbenzyl) diselane may be incorporated into one or more polymers, with the polymer(s) serving as a substrate for carrying/supporting the organo-diselenide compound for utilization as an agent against infectious, disease-causing agents, whether it is in the prevention of infestation, inhibition of growth and/or spread, and/or the treatment of infested or contaminated surface(s) and/or articles.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the U.S. Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. It is intended that the application is defined by the claims appended hereto.

What is claimed is:

1. A composition comprising an organic diselenide having the following formula:

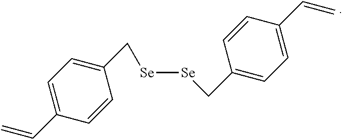 [SMILES: C=Cc1ccc(C[Se][Se]Cc2ccc(C=C)cc2)cc1]

2. A method for preventing and/or treating a surface with a biocidal agent comprising an organic diselenide having the following formula:

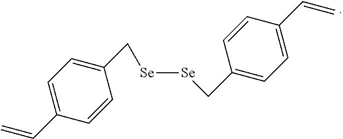 [SMILES: C=Cc1ccc(C[Se][Se]Cc2ccc(C=C)cc2)cc1]

3. A composition for incorporation with one or more polymers through a chemical bonding process, the composition including one or more polymers as a substrate supporting an organic diselenide having the following formula: 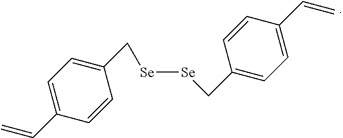 [SMILES: C=Cc1ccc(C[Se][Se]Cc2ccc(C=C)cc2)cc1]
